(19) United States
(12) Patent Application Publication
(10) Pub. No.: US 2019/0069928 A1
(43) Pub. Date: Mar. 7, 2019

(54) INJECTION NEEDLE FOR ENDOSCOPE

(71) Applicant: OLYMPUS CORPORATION, Tokyo (JP)

(72) Inventors: Kensuke UESAKA, Tokyo (JP); Koji TOMINAGA, Tokyo (JP)

(73) Assignee: OLYMPUS CORPORATION, Tokyo (JP)

(21) Appl. No.: 16/178,758

(22) Filed: Nov. 2, 2018

Related U.S. Application Data
(63) Continuation of application No. PCT/JP2016/064588, filed on May 17, 2016.

(51) Int. Cl. A61B 17/34; A61M 5/158

(52) U.S. Cl. CPC A61B 17/3478; A61B 1/0008; A61M 5/158

(57) ABSTRACT

An injection needle for an endoscope includes: an outer and inner sheath; a needle tube; a stopper; an engaging contact portion integrally formed of the same material as the outer sheath, protrudes to an inner side in a radial direction relative to an inner circumferential surface of the outer sheath; and an operating member. A movable range of the operating member in a straight state is made up of a first range and a second range. A length of the second range is greater than a value of a difference between an inner diameter of the outer sheath and an outer diameter of the inner sheath, and a thickness of the engaging contact portion in the direction of the longitudinal axis is greater than a thickness of the inner sheath in a radial direction and a thickness of the outer sheath in the radial direction.

This application is a continuation application based on PCT Patent Application No. PCT/JP2016/064588, filed May 17, 2016, the content of which is incorporated herein by reference.

### BACKGROUND OF THE INVENTION

### Field of the Invention

The present invention relates to an injection needle for an endoscope which is introduced into a body cavity through a channel of the endoscope for usage.

### Description of the Related Art

Conventionally, an injection needle for an endoscope introduced into a body cavity through a channel of the endoscope and used to perform injection into tissue in the body cavity is known.

For example, in an injection needle for an endoscope disclosed in Japanese Unexamined Patent Application, First Publication No. 2001-58006, a needle body is mounted on a distal end of an inner tube, the inner tube is configured to be advanceable and retractable in an outer tube by manipulating a operating portion, and the needle body is projected from a distal end of the outer tube by pushing the inner tube inside the outer tube toward a side close to the distal end and punctures tissue in a body cavity. In the injection needle for an endoscope disclosed in Japanese Unexamined Patent Application, First Publication No. 2001-58006, a small-diameter portion that fills a clearance between an outer circumference of the needle body and an inner circumferential surface of the inner tube is provided, and thereby the inner tube having a certain diameter is commonly usable regardless of a difference of an outer diameter of the needle body.

In the injection needle for an endoscope, a length of the inner tube is set to project the needle body from the distal end of the outer tube. Since the injection needle for an endoscope is inserted into an insertion portion of the endoscope that meanders in the body cavity, the outer tube and the inner tube also meander. When the outer tube and the inner tube meander in this way, a variation of path length of the inner tube in the outer tube occurs, and a protrusion length at which the needle body protrudes from an opening of the distal end of the outer tube sometimes varies.

Furthermore, an active curvature portion of the insertion portion of the endoscope is greatly curved in a state in which the injection needle for an endoscope protrudes from an opening of a distal end of the channel of the endoscope, and thus the outer tube and the inner tube are sometimes greatly curved. In this case, the protrusion length of the needle body also varies.

When a meander is strong or large curvature occurs, a problem in which a distal end of the needle body does not reach the distal end of the outer tube or a problem in which an amount of protrusion of the needle body from the distal end of the outer tube is not sufficiently obtained occurs.

In the injection needle for an endoscope disclosed in Japanese Unexamined Patent Application, First Publication No. 2001-58006, a stroke difference of the inner tube from the outer tube is increased such that the needle body can sufficiently protrude from the distal end of the outer tube. Furthermore, in the injection needle for an endoscope of Japanese Unexamined Patent Application, First Publication No. 2001-58006, a large-diameter portion is fixed to the outer circumference of the needle body at a side closer to the distal end than the small-diameter portion. A distal end face of the large-diameter portion comes into contact with an inner end face of an engaging contact portion provided in the opening of the distal end of the outer tube. Thereby, the amount of protrusion of the needle body from the outer tube is regulated, and the needle body can be prevented from excessively protruding from the outer tube and puncturing the tissue to an excessive depth.

### SUMMARY OF THE INVENTION

An injection needle for an endoscope according to a first aspect of the present invention includes: an outer sheath configured to be inserted through a channel of the endoscope; an inner sheath inserted through the outer sheath to be movable advance-retract; a needle tube disposed at a distal end portion of the inner sheath and configured to have a distal end portion that is caused to protrude from a distal end of the outer sheath by the advance movement of the inner sheath and to communicate with a lumen of the inner sheath; a stopper disposed between the distal end portion of the inner sheath and the distal end portion of the needle tube; an engaging contact portion integrally formed with the outer sheath and formed of the same type of material as the outer sheath, and configured to protrude to an inner side in a radial direction relative to an inner circumferential surface of the outer sheath at a distal end portion of the outer sheath and to be able to come into contact with the stopper; and an operating member connected to a proximal end portion of the inner sheath, moved in a direction of a longitudinal axis of the inner sheath by manipulation of an operator, and configured to move the inner sheath advance-retract relative to the outer sheath. A movable range of the operating member in a straight state in which the outer sheath and the inner sheath extend in a straight shape in the direction of the longitudinal axis is made up of a first range in which the operating member is moved in a state in which the stopper is separated from the engaging contact portion, and a second range in which the operating member is moved in a state in which the stopper is in contact with the engaging contact portion. A length of the second range is larger than a value of a difference between an inner diameter of the outer sheath and an outer diameter of the inner sheath, and a thickness of the engaging contact portion in the direction of the longitudinal axis is greater than a thickness of the inner sheath in a radial direction and a thickness of the outer sheath in the radial direction.

A second aspect of the present invention may be configured such that, in the injection needle for an endoscope according to the first aspect, the outer sheath and the inner sheath may be formed of the same type of resin material, and the thickness of the outer sheath in the radial direction may be greater than the thickness of the inner sheath in the radial direction.

A third aspect of the present invention may be configured such that, in the injection needle for an endoscope according to the first aspect, the proximal face of the engaging contact portion may be orthogonal to the longitudinal axis of the inner sheath.

An injection needle for an endoscope according to a fourth aspect of the present invention includes: an outer sheath configured to be inserted through a channel of the endoscope; an inner sheath inserted through the outer sheath to be advanceable and retractable; a needle tube disposed at a distal end portion of the inner sheath and configured to have a distal end portion that is caused to protrude from a distal end of the outer sheath by the advance of the inner sheath; a stopper disposed between the distal end portion of the inner sheath and the distal end portion of the needle tube; an engaging contact portion integrally formed with the outer sheath and formed of the same type of material as the outer sheath, the engaging contact portion being configured to protrude to an inner side in a radial direction relative to an inner circumferential surface of the outer sheath at a distal end portion of the outer sheath and to have a proximal end face that is capable of coming into contact with the stopper and a distal end face which faces a distal side of the outer sheath; and an operating member connected to a proximal end portion of the inner sheath, and configured to move the inner sheath to the distal side of the outer sheath by movement to the distal side of the outer sheath and to be further movable to the distal side of the outer sheath by a predetermined length in a state in which the stopper is in contact with the engaging contact portion, wherein the predetermined length in the operating member is larger than a value of a difference between an inner diameter of the outer sheath and an outer diameter of the inner sheath, a thickness of the engaging contact portion in a radial direction is greater than a thickness of the outer sheath in the radial direction from the proximal end face to the distal end face of the engaging contact portion, a value of a length from the proximal end face to the distal end face of the engaging contact portion is greater than a value of the thickness of the inner sheath in the radial direction and a value of the thickness of the outer sheath in the radial direction, and the operating member is capable of causing the inner sheath to be pushed toward a distal side in a state in which the inner sheath meanders due to a compressive force in the direction of the longitudinal axis and comes into contact with the inner circumferential surface of the outer sheath.

A fifth aspect of the present invention may be configured such that, in the injection needle for an endoscope according to the fourth aspect, the outer sheath and the inner sheath may be formed of the same type of resin material; and the thickness of the outer sheath in the radial direction may be greater than the thickness of the inner sheath in the radial direction.

A sixth aspect of the present invention may be configured such that, in the injection needle for an endoscope according to the fourth aspect, the proximal end face of the engaging contact portion may be made up of a face that is orthogonal to the longitudinal axis of the inner sheath.

### BRIEF DESCRIPTION OF DRAWINGS

FIG. 1 is an overall view of an injection needle for an endoscope according to an embodiment of the present invention.

FIG. 2 is a view illustrating a state in which a needle tube of the injection needle for an endoscope according to the embodiment of the present invention is housed in an outer sheath, and a view illustrating a distal end portion of the injection needle for an endoscope in a sectional view of a longitudinal direction.

FIG. 3 is a schematic view illustrating a constitution of the injection needle for an endoscope according to the embodiment of the present invention.

FIG. 4 is a view illustrating a state in which the needle tube of the injection needle for an endoscope according to the embodiment of the present invention protrudes from a distal end of the outer sheath, and a view illustrating the distal end portion of the injection needle for an endoscope in the sectional view of the longitudinal direction.

FIG. 5 is a view illustrating a state in which the needle tube of the injection needle for an endoscope according to the embodiment of the present invention protrudes from the distal end of the outer sheath, and a view illustrating the distal end portion of the injection needle for an endoscope in the sectional view of the longitudinal direction.

FIG. 6 is a view illustrating an aspect of using the injection needle for an endoscope according to the embodiment of the present invention.

### DETAILED DESCRIPTION OF THE INVENTION

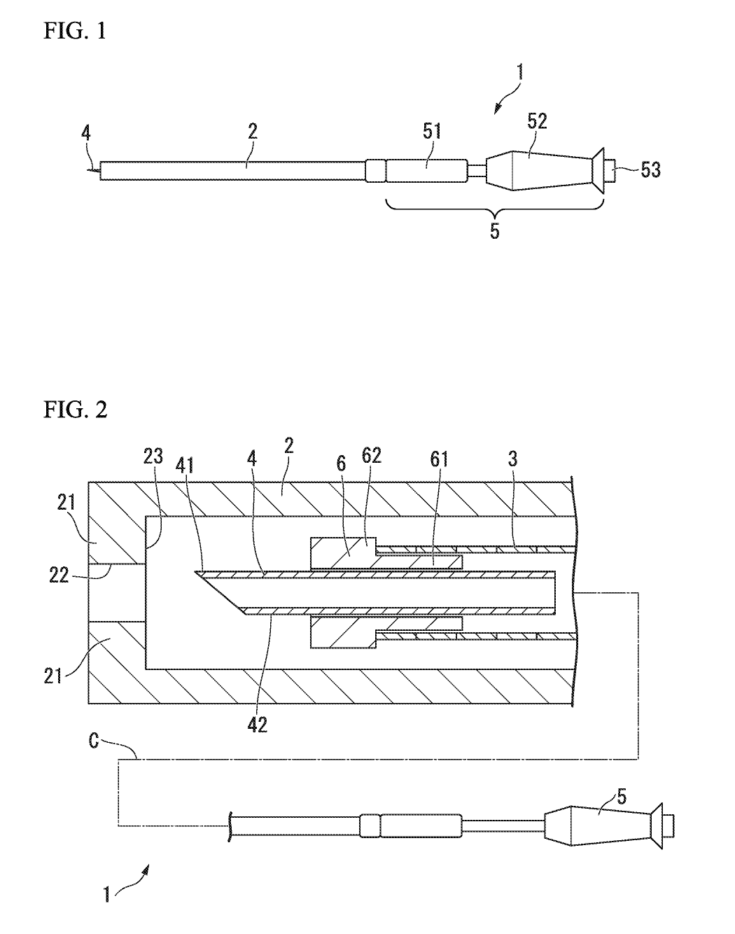

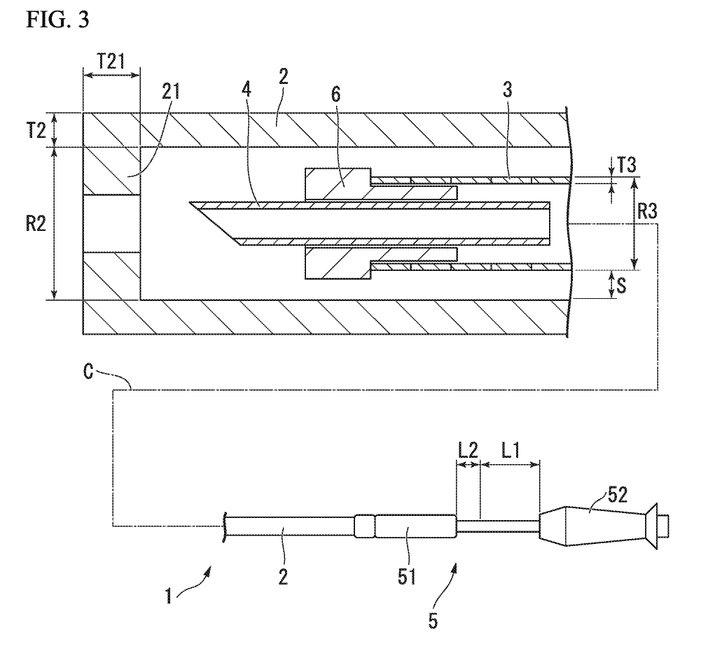

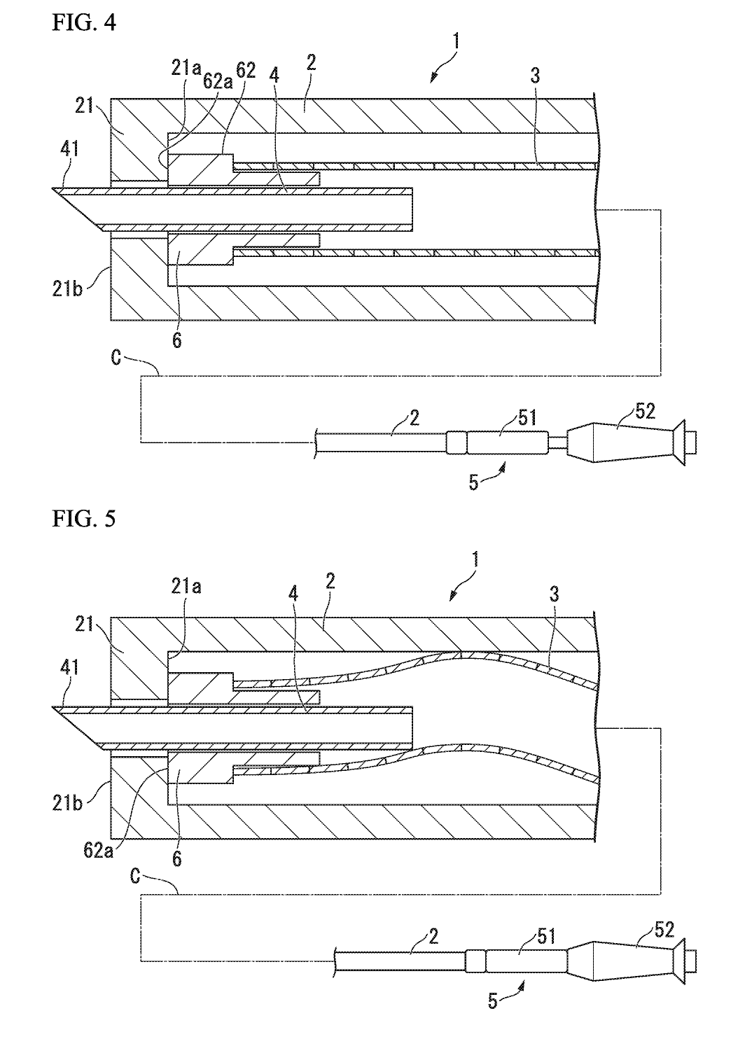

An embodiment of the present invention will be described with reference to FIGS. 1 to 6. FIG. 1 is an overall view of an injection needle 1 for an endoscope according to an embodiment of the present invention. FIG. 2 is a view illustrating a state in which a needle tube 4 of the injection needle 1 for an endoscope is housed in an outer sheath 2, and a view illustrating a distal end portion of the injection needle 1 for an endoscope in a sectional view of a direction of a longitudinal axis C. FIG. 3 is a schematic view illustrating a constitution of the injection needle 1 for an endoscope. FIGS. 4 and 5 are views illustrating a state in which the needle tube 4 of the injection needle 1 for an endoscope protrudes from a distal end of the outer sheath 2, and a view illustrating the distal end portion of the injection needle 1 for an endoscope in a sectional view of a longitudinal direction.

As illustrated in FIGS. 1 and 2, the injection needle 1 for an endoscope according to the present embodiment includes an outer sheath 2, an inner sheath 3, a needle tube 4, a stopper 6, and an operating portion 5, and is inserted through a channel of the endoscope for usage. The outer sheath 2 and the inner sheath 3 are elongated tubes so as to be flexibly deformable following deformation of the channel of the endoscope.

The outer sheath 2 has a lumen formed over the entire length of the outer sheath 2 from a distal end thereof to a proximal end thereof. As illustrated in FIG. 2, an engaging contact portion 21 that protrudes to an inner side of the outer sheath 2 in a radial direction relative to an inner circumferential surface of the outer sheath 2 is formed at a distal end portion of the outer sheath 2. The engaging contact portion 21 has an approximately annular surface that is orthogonal to a direction of the longitudinal axis C of the outer sheath 2, and has a distal end opening 22 formed in the center of the engaging contact portion 21. The distal end opening 22 has an opening diameter that is slightly greater than an outer diameter of the needle tube 4, and is configured such that the needle tube 4 is capable of protruding from the distal end of the outer sheath 2. A surface of the engaging contact portion 21 at their proximal side is configured to be able to come into contact with the stopper 6. A thickness of the engaging contact portion 21 in the direction of the longitudinal axis C is formed to be greater than a thickness of the outer sheath 2 in the radial direction.

The engaging contact portion 21 is integrally formed with the outer sheath 2 and formed of the same type of resin material as the outer sheath 2. For example, the outer sheath 2 and the engaging contact portion 21 are formed of a resin or the like of a fluorine tube or a nylon tube. An example of the material of which the outer sheath 2 and the engaging contact portion 21 are formed includes polytetrafluoroethylene (PTFE) or perfluoroalkoxy alkane (PFA). In the present embodiment, the engaging contact portion 21 is formed integrally with the outer sheath 2 by thermoforming.

The inner sheath 3 has a lumen formed over the entire length of the inner sheath 3 from a distal end thereof to a proximal end thereof. The inner sheath 3 has a smaller outer diameter than an inner diameter of the outer sheath 2, and a gap is formed between an outer circumferential surface of the inner sheath 3 and an inner circumferential surface of the lumen of the outer sheath 2. A thickness of the inner sheath 3 in the radial direction is smaller than that of the outer sheath 2 in the radial direction. A proximal end portion of the inner sheath 3 is connected to an operating member 52 (to be described below). The needle tube 4 is mounted on a distal end portion of the inner sheath 3.

Like the outer sheath 2, the inner sheath 3 is formed of a resin or the like e.g., a fluorine tube or a nylon tube. An example of the material of which the inner sheath 3 is formed includes polytetrafluoroethylene (PTFE) or perfluoroalkoxy alkane (PFA). In the present embodiment, the outer sheath 2 and the inner sheath 3 are formed of the same materials whose Young's moduli are equal to each other.

“The inner sheath 3 and the outer sheath 2 are formed of the same type resin materials” includes a case that the inner sheath and the outer sheath are formed of the resin material whose Young's moduli are substantially equivalent to each other. For example, in the case of a resin whose Young's modulus ranges from 0.3 to 0.6 GPa, a combination of the inner sheath 3 formed of PTFE and the outer sheath 2 formed of PFA may be made.

The needle tube 4 is a tubular member in which a lumen is formed over the entire length of the needle tube 4 from a distal end thereof to a proximal end thereof, and has a needle tip 41 that is obliquely cut in a direction that intersects the longitudinal axis C and is formed in a sharp blade shape at a distal end portion 42 of the needle tube 4.

As illustrated in FIG. 2, the stopper 6 is a cylindrical member having a communication hole that is communicated in the direction of the longitudinal axis C, and has a small-diameter portion 61 close to a proximal end thereof and a large-diameter portion 62 close to a distal end thereof. The small-diameter portion 61 has an outer diameter that is slightly smaller than an inner diameter of the lumen of the inner sheath 3. The large-diameter portion 62 is enlarged in diameter at a side closer to the distal end than the small-diameter portion 61, has a diameter that is larger than or equal to the outer diameter of the inner sheath 3, and has a larger outer diameter than the diameter of the distal end opening 22 of the engaging contact portion 21.

The needle tube 4 is inserted through the communication hole of the stopper 6. The distal end portion 42 protrudes to the distal side than the large-diameter portion 62 of the stopper 6, and a proximal end of the needle tube 4 protrudes closer to the proximal end than the small-diameter portion 61. The needle tube 4 is fixed to the stopper 6 by caulking.

The small-diameter portion 61 of the stopper 6 is inserted through the distal end portion of the inner sheath 3, and is fixed to the inner sheath 3 by an adhesive. Therefore, the needle tube 4 and the inner sheath 3 are connected via the stopper 6. The large-diameter portion 62 of the stopper 6 is disposed between the distal end portion of the inner sheath 3 and the distal end portion 42 of the needle tube 4. The distal end portion 42 of the needle tube 4 connected to the inner sheath 3 is disposed more distal side than the distal end of the inner sheath 3. The proximal end portion of the needle tube 4 is inserted into the lumen of the distal end portion of the inner sheath 3. The lumen of the needle tube 4 communicates with the lumen of the inner sheath 3.

The inner sheath 3 is disposed such that the distal end portion 42 of the needle tube 4 is capable of protruding from the distal end opening 22 of the engaging contact portion 21 (from the distal end of the outer sheath 2) in accordance with advance-retract of the inner sheath 3 relative to the outer sheath 2. To be specific, a length of the inner sheath 3 that is capable of advancing and retracting in the outer sheath 2 is set to be longer than the entire length of the outer sheath 2 such that the distal end portion 42 of the needle tube 4 is capable of protruding from the distal end of the outer sheath 2 even when a variation of path lengths of the outer sheath 2 and the inner sheath 3 occurs in accordance with curvature of the outer sheath 2.

The operating portion 5 is provided close to the proximal end of the outer sheath 2. The operating portion 5 includes an operating main body 51 and an operating member 52. The operating main body 51 is fixed to the proximal end of the outer sheath 2. The operating member 52 is mounted on the proximal end portion of the inner sheath 3 via a pipe (not shown) formed of stainless steel, and is provided to be advanceable and retractable relative to the operating main body 51 in the direction of the longitudinal axis C. A proximal end channel port 53 is provided at a proximal end portion of the operating member 52. The proximal end channel port 53 is configured such that a syringe (not shown) is capable of being attached. The operating portion 5 is configured such that the inner sheath 3 advances and retractes relative to the outer sheath 2 by the advance-retract of the operating member 52 relative to the operating main body 51.

A movable range of the operating member 52 in a straight state in which the outer sheath 2 and the inner sheath 3 extend in a straight shape along the direction of the longitudinal axis C is divided into a first range and a second range.

The first range is a range in which the operating member 52 moves in a state in which the stopper 6 is separated from the engaging contact portion 21 to a proximal side. The second range is a range in which the operating member 52 moves in a state in which the stopper 6 is in contact with the engaging contact portion 21. That is, the first range is a length L1 in the straight state by which the operating member 52 is pushed toward a distal side for advancing the inner sheath 3 from a state in which the inner sheath 3 is located at the most proximal side to a state in which the inner sheath 3 advances and a distal end face 62a of the stopper 6 comes into contact with a proximal end face 21a of the engaging contact portion 21. The distal end face 21b of of the engaging contact portion 21 faces a distal side of the outer sheath 2.

In a state in which the outer sheath 2 and the inner sheath 3 are curved, the path length of the inner sheath 3 in the outer sheath 2 might be varied. When the outer sheath 2 and the inner sheath 3 are curved, the inner sheath 3 comes into contact with an inner wall of the lumen of the outer sheath 2, and friction occurs between the inner sheath 3 and the outer sheath 2, as a result, the inner sheath 3 might be not moved smoothly sometimes. In this case, if an amount of the advance-retract movement of the inner sheath 3 relative to the outer sheath 2 is set to be equal to an amount by which the distal end portion 42 of the needle tube 4 can protrude from the distal end of the outer sheath 2 in the straight state, the distal end portion 42 of the needle tube 4 does not reach a position at which the needle tube 4 protrudes from the distal end of the outer sheath 2 sometimes even if the operating member is pushed in the first range. Thus, the amount of the advance-retract of the inner sheath 3 relative to the outer sheath 2 is set to be long such that the needle tip 41 reliably protrudes from the distal end of the outer sheath 2 even in a state in which the outer sheath 2 and the inner sheath 3 are curved.

As described above, a length of the inner sheath 3 that is capable of advancing and retracting in the outer sheath 2 is set to be longer than the entire length of the outer sheath 2. For this reason, in the straight state, even after the distal end face 62a of the stopper 6 comes into contact with the proximal end face 21a of the engaging contact portion 21, the operating member 52 is capable of being pushed toward a distal side. A range in which the operating member 52 is capable of being pushed toward the distal side even after the distal end face 62a of the stopper 6 comes into contact with the proximal end face 21a of the engaging contact portion 21 in the straight state is referred to as the second range. A force that is compressed in the lumen of the outer sheath 2 in the direction of the longitudinal axis C acts on the inner sheath 3 in the second range, so that the inner sheath 3 is elastically compressed and simultaneously, as illustrated in FIG. 5, the inner sheath 3 meanders in the lumen of the outer sheath 2.

Since the amount of the advance-retract of the inner sheath 3 relative to the outer sheath 2 assumes a case in which an amount of curvature is large, the operating member 52 is capable of being pushed after the stopper 6 comes into contact with the engaging contact portion 21 even when the curvature of the sheath is small without being limited to the straight state. Here, the case in which the amount of curvature is large includes a case in which the curvature occurs in a plurality of places of the outer sheath 2 and the inner sheath 3 and a total thereof is large, in addition to a case in which a curvature of the curved portion is large in one place of the outer sheath 2 and the inner sheath 3.

Next, a dimensional relationship of each portion of the injection needle 1 for an endoscope will be described with reference to FIG. 3.

As illustrated in FIG. 3, a thickness T21 of the engaging contact portion 21 in the direction of the longitudinal axis C is greater than a thickness T3 of the inner sheath 33 in the radial direction and a thickness T2 of the outer sheath 2 in the radial direction. Since the engaging contact portion 21 and the outer sheath 2 are integrally formed of the same type of material, the thickness T21 of the engaging contact portion 21 is made greater than the thickness T2 of the outer sheath 2, and thereby strength of the engaging contact portion 21 is capable of being be made higher than that of an outer circumferential portion of the outer sheath 2.

In the conventional injection needle for an endoscope, the thickness of the engaging contact portion in the direction of the longitudinal axis was set to be nearly equal to the thickness of the outer sheath. For this reason, when the inner sheath was further pushed toward the distal side in the state in which the stopper was in contact with the engaging contact portion, and a great force was applied to the engaging contact portion, the engaging contact portion and the distal end portion of the outer sheath was bent outward by a load toward the engaging contact portion, and there was a concern that the stopper protruded to the more distal than the outer sheath. Therefore, there was a need to inhibit an amount of extrusion of the inner sheath.

In the injection needle 1 for an endoscope according to the present embodiment, even when the inner sheath 3 is further pushed toward the distal side in the state in which the stopper 6 is in contact with the engaging contact portion 21, and a great force is applied to the engaging contact portion 21, the advance of the stopper 6 is capable of being reliably regulated by the engaging contact portion 21. Therefore, an amount of extrusion of the inner sheath 3 is capable of being set to be large.

Since the thickness T21 of the engaging contact portion 21 is greater than the thickness T3 of the inner sheath 3, the strength of the engaging contact portion 21 is made higher than that of the inner sheath 3. Therefore, the inner sheath 3 is compressed in the outer sheath 2 first, and meanders in the outer sheath 2 in the state in which the stopper 6 is in contact with the engaging contact portion 21. As a result, even when the inner sheath 3 is further pushed toward the further distal side in the state in which the stopper 6 is in contact with the engaging contact portion 21, and a great force is applied to the engaging contact portion 21, the stopper 6 is prevented from breaking through the engaging contact portion 21.

The length (the stroke length) L2 of the second range of the operating member 52 is greater than a value of a difference between an inner diameter R2 of the outer sheath 2 and an outer diameter R3 of the inner sheath 3. The difference between the inner diameter R2 of the outer sheath 2 and the outer diameter R3 of the inner sheath 3 is a clearance S between the inner circumferential surface of the outer sheath 2 and the outer circumferential surface of the inner sheath 3. Meanders of the inner sheath 3 is capable of being inhibited by reducing the clearance S between the inner diameter R2 of the outer sheath 2 and the outer diameter R3 of the inner sheath 3. By inhibiting the meanders of the inner sheath 3, the variation of the path lengths of the outer sheath 2 and the inner sheath 3 in accordance with the curvature of the outer sheath 2 is capable of being inhibited.

Furthermore, the length L2 of the second range of the operating member 52 is greater than the clearance S. The length L2 of the second range is greater than the clearance S. As a result, as illustrated in FIG. 5, in the straight state, even when the inner sheath 3 meanders in the outer sheath 2 at least once in the state in which the stopper 6 is in contact with the engaging contact portion 21, if the operating member 52 is pushed toward a further distal side, the distal end portion 42 of the needle tube 4 is capable of being reliably projected from the distal end of the outer sheath 2. That is, the injection needle 1 for an endoscope according to the present embodiment has an effect of enabling the needle tube 4 to protrude to the further distal side than the distal end of the outer sheath 2 up to a desired length while inhibiting the meanders of the inner sheath 3.

In an actual specification mode, when the possibility of the inner sheath 3 meandering in a plurality of places in the outer sheath 2 is taken into consideration and, for instance, the clearance S is 0.2 mm whereas the length L2 of the second range of the operating member 52 is set to 8 mm, the needle tip 41 is capable of being reliably projected from the distal end of the outer sheath 2.

Furthermore, the thickness T2 of the outer sheath 2 may be set to be larger than the thickness T3 of the inner sheath 3. As the thickness T2 of the outer sheath 2 is set to be larger than the thickness T3 of the inner sheath 3, the strength of the outer circumferential portion of the outer sheath 2 is made higher than that of the inner sheath 3. As a result, when the inner sheath 3 is further pushed toward the distal side in the state in which the stopper 6 is in contact with the engaging contact portion 21, and a great force is applied to the engaging contact portion 21, the inner sheath 3 is configured to be compressed first.

That is, as illustrated in FIG. 4, when the inner sheath 3 is further pushed toward the distal side in the state in which the stopper 6 is in contact with the engaging contact portion 21, the inner sheath 3 is compressed first as illustrated in FIG. 5. When the inner sheath 3 is further pushed toward the distal side, the outer sheath 2 extends next in the direction of longitudinal axis. Such a force in which the engaging contact portion 21 is deformed is applied to the engaging contact portion 21 after the compression of the inner sheath 3 and the extension of the outer sheath 2. Therefore, when the thickness T2 of the outer sheath 2 is set to be larger than the thickness T3 of the inner sheath 3, an additional effect in which the engaging contact portion 21 is capable of regulating the advance movement of the stopper 6 even if the thickness T21 of the engaging contact portion 21 is not set to be larger than necessary can be produced. Accordingly, an increase in the amount of the advance-retract movement of the operating member due to an increase in the thickness T21 of the engaging contact portion 21 is capable of being inhibited.

Since the inner sheath 3 is capable of being pushed toward the distal side with a great force in the state in which the stopper 6 is in contact with the engaging contact portion 21, for instance even if the needle tip 41 is punctured into a hard organ, the needle tube 4 is capable of being punctured without being pushed back to a proximal side.

For example, when the outer sheath 2 and the inner sheath 3 are formed of the same material, the thickness T2 of the outer sheath 2 is set to 0.325 mm to 0.425 mm, the thickness T21 of the engaging contact portion 21 is set to 0.5 mm to 0.7 mm, the thickness T3 of the inner sheath 3 is set to 0.25 mm to 0.35 mm, the clearance S between the inner diameter of the outer sheath 2 and the outer diameter of the inner sheath 3 is set to 0.05 mm to 0.15 mm, and the length L2 of the second range of the operating member is set to 6 mm to 10 mm, a relationship among their sizes needs only to satisfy the above conditions.

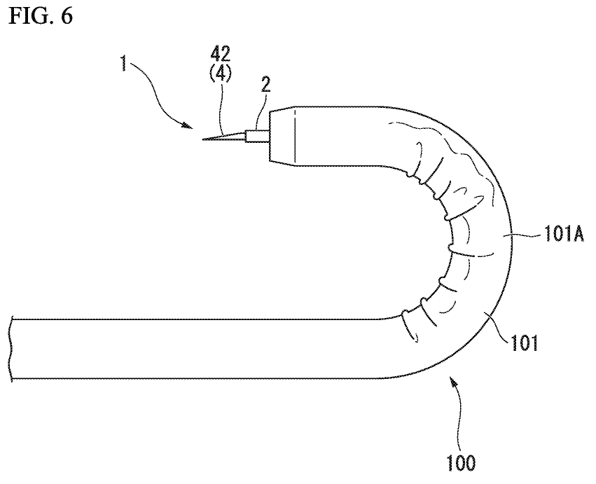

An aspect of using the injection needle 1 for an endoscope according to the present embodiment will be described. FIG. 6 is a view illustrating an aspect of using the injection needle 1 for an endoscope according to the present embodiment.

The injection needle 1 for an endoscope is inserted into the body via a channel (not shown) of an inserting portion 101 of a well-known endoscope 100.

An active curvature portion 101A is configured to be curved in a predetermined direction by pulling an angle wire (not shown), which is fixed to a distal end of the active curvature portion 101A and extends up to an endoscope operating portion, in the operating portion.

As illustrated in FIG. 6, as the active curvature portion 101A provided at the inserting portion 101 is strongly curved during the use of the endoscope 100, the injection needle 1 for an endoscope is also bent. In the injection needle 1 for an endoscope according to the present embodiment, even in a state in which the outer sheath 2 is strongly bent, the inner sheath 3 is smoothly advanced, and the distal end portion 42 of the needle tube 4 is reliably projected from the distal end of the outer sheath 2, so that the needle tip 41 is configured to be capable of being punctured into tissue T.

As described above, according to the injection needle 1 for an endoscope of the present embodiment, the range in which the operating member 52 is movable in the straight state in which the outer sheath 2 and the inner sheath 3 extend in the straight shape in the direction of the longitudinal axis C is made up of the first range and the second range, the length L2 of the second range is larger than the value of the difference between the inner diameter R2 of the outer sheath 2 and the outer diameter R3 of the inner sheath 3, and the thickness T21 of the engaging contact portion 21 is greater than the thickness T3 of the inner sheath 3 and the thickness T2 of the outer sheath 2. As a result, when the outer sheath 2 and the inner sheath 3 are curved, the meanders of the inner sheath 3 in the outer sheath 2 are inhibited, and frictional resistance of the inner sheath 3 to the outer sheath 2 is inhibited, so that the inner sheath 3 is smoothly advanced toward the distal side. When the inner sheath 3 is pushed toward the distal side in the state in which the stopper 6 is in contact with the engaging contact portion 21, additional advance of the inner sheath 3 from the engaging contact portion 21 is capable of being reliably inhibited.

According to the injection needle 1 for an endoscope of the present embodiment, since the thickness T2 of the outer sheath 2 in the radial direction is greater than the thickness T3 of the inner sheath 3 in the radial direction, the strength of the outer circumferential portion of the outer sheath 2 is made higher than that of the inner sheath 3. As a result, when the operating member 52 is pushed toward the distal side in the state in which the stopper 6 is in contact with the engaging contact portion 21, the inner sheath 3 is configured to be compressed first.

The injection needle 1 for an endoscope according to the present embodiment is favorably used when the outer sheath 2 is curved in accordance with the strong curvature of the active curvature portion 101A provided at the inserting portion 101 of the endoscope 100. In addition, the injection needle 1 also is favorably used when the entire length of the sheath is long and the meander portions are increased, for example, when a procedure is performed by inserting the endoscope into the colon from the anus.

Although the embodiment of the present invention has been described above in detail with reference to the drawings, the specific constitution is not limited to the embodiment, and also includes a change in design, etc. without departing from the scope of the present invention. Further, the components represented in the embodiment can be configured by an appropriate combination.

What is claimed is:

1. An injection needle for an endoscope, comprising:
an outer sheath configured to be inserted through a channel of the endoscope;
an inner sheath inserted through the outer sheath to be advanceable and retractable;
a needle tube disposed at a distal end portion of the inner sheath and configured to have a distal end portion that is caused to protrude from a distal end of the outer sheath by the advance of the inner sheath, the needle tube communicating with a lumen of the inner sheath;
a stopper disposed between the distal end portion of the inner sheath and the distal end portion of the needle tube;
an engaging contact portion integrally formed with the outer sheath and formed of the same type of material as the outer sheath, the engaging contact portion being configured to protrude to an inner side in a radial direction relative to an inner circumferential surface of the outer sheath at a distal end portion of the outer sheath, the engaging contact portion including a proximal end face configured to be capable of coming into contact with the stopper, and a distal end face faced to a distal side of the outer sheath; and
an operating member connected to a proximal end portion of the inner sheath, configured to be moved in a direction of a longitudinal axis of the inner sheath by manipulation of an operator, and configured to advance and retract the inner sheath relative to the outer sheath,
wherein a movable range of the operating member in a straight state in which the outer sheath and the inner sheath extend in a straight shape along the direction of the longitudinal axis is made up of
a first range in which the operating member is moved in a state in which the stopper is separated from the engaging contact portion, and
a second range in which the operating member is moved in a state in which the stopper is in contact with the engaging contact portion,
a length of the second range is larger than a value of a difference between an inner diameter of the outer sheath and an outer diameter of the inner sheath,
a thickness of the engaging contact portion in the direction of the longitudinal axis is greater than a thickness of the inner sheath in a radial direction and a thickness of the outer sheath in the radial direction,
a value of a length from the proximal face to the distal face of the engaging contact portion is greater than a value of the thickness of the inner sheath in the radial direction and a value of the thickness of the outer sheath in the radial direction, and
the operating member is capable of causing the inner sheath to be pushed toward a distal side in a state in which the inner sheath meanders due to a compressive force in the direction of the longitudinal axis and comes into contact with the inner circumferential surface of the outer sheath.

2. The injection needle for an endoscope according to claim 1, wherein:
the outer sheath and the inner sheath are formed of the same type of resin material; and
the thickness of the outer sheath in the radial direction is greater than the thickness of the inner sheath in the radial direction.

3. The injection needle for an endoscope according to claim 1, wherein the proximal face of the engaging contact portion is orthogonal to the longitudinal axis of the inner sheath.

4. An injection needle for an endoscope, comprising:
an outer sheath configured to be inserted through a channel of the endoscope;
an inner sheath inserted through the outer sheath to be advanceable and retractable;
a needle tube disposed at a distal end portion of the inner sheath and configured to have a distal end portion that is caused to protrude from a distal end of the outer sheath by the advance of the inner sheath;
a stopper disposed between the distal end portion of the inner sheath and the distal end portion of the needle tube;
an engaging contact portion integrally formed with the outer sheath and formed of the same type of material as the outer sheath, the engaging contact portion configured to protrude to an inner side in a radial direction relative to an inner circumferential surface of the outer sheath at a distal end portion of the outer sheath and to have a proximal end face that is capable of coming into contact with the stopper and a distal end face which faces a distal side of the outer sheath; and
an operating member connected to a proximal end portion of the inner sheath, and configured to move the inner sheath to the distal side of the outer sheath by movement to the distal side of the outer sheath and to be further movable to the distal side of the outer sheath by a predetermined length in a state in which the stopper is in contact with the engaging contact portion,
wherein the predetermined length in the operating member is larger than a value of a difference between an inner diameter of the outer sheath and an outer diameter of the inner sheath,
a thickness of the engaging contact portion in a radial direction is greater than a thickness of the outer sheath in the radial direction from the proximal end face to the distal end face of the engaging contact portion,
a value of a length from the proximal end face to the distal end face of the engaging contact portion is greater than a value of the thickness of the inner sheath in the radial direction and a value of the thickness of the outer sheath in the radial direction, and
the operating member is capable of causing the inner sheath to be pushed toward a distal side in a state in which the inner sheath meanders due to a compressive force in the direction of the longitudinal axis and comes into contact with the inner circumferential surface of the outer sheath.

5. The injection needle for an endoscope according to claim 4, wherein:
the outer sheath and the inner sheath are formed of the same type of resin material; and
the thickness of the outer sheath in the radial direction is greater than the thickness of the inner sheath in the radial direction.

6. The injection needle for an endoscope according to claim 4, wherein the proximal end face of the engaging contact portion is made up of a face that is orthogonal to the longitudinal axis of the inner sheath.
